(19) United States
(12) Patent Application Publication
(10) Pub. No.: US 2026/0041439 A1
(43) Pub. Date: Feb. 12, 2026

(54) MEDICAL MOTOR HANDPIECE FOR 2-IN-1 OPERATION AND MEDICAL MANUAL INSTRUMENT WITH 2-IN-1 OPERATION

(71) Applicant: Aesculap AG, Tuttlingen (DE)

(72) Inventors: Andre Buerk, Villingen-Schwenningen (DE); Thomas Hagen, Tuttlingen (DE); Simone Hermle, Villingen-Schwenningen (DE); Lilian Killinger, Ehningen (DE); Juergen Barth, Denkingen (DE); Roland-Alois Hoegerle, Tuttlingen (DE); Uwe Schaz, Neuhausen (DE); Aaron Vogler, Leibertingen (DE)

(21) Appl. No.: 19/102,190

(22) PCT Filed: Aug. 3, 2023
(86) PCT No.: PCT/EP2023/071589

§ 371 Date: Feb. 7, 2025

(30) Foreign Application Priority Data
Aug. 9, 2022 (DE) 10 2022 119 979.7

(51) Int. Cl. A61B 17/16; A61B 17/00

(52) U.S. Cl. CPC A61B 17/1624; A61B 17/00234; A61B 17/162

(57) ABSTRACT

A medical motor handpiece for driving a distal end effector includes a handle portion and a preferably sleeve-shaped operating element that is held to rotate about a handle longitudinal axis at a distal end portion of the handle portion. The operating element can be coupled to the end effector so as to transform a rotational movement of the operating element in a first direction of rotation into a movement of the end effector. In the event of a rotational movement of the operating element in a second direction of rotation, opposite the first direction of rotation, a function on the end effector can be effected. The medical motor handpiece can be a component of a medical manual instrument that has an end effector coupled to the medical motor handpiece via a shaft in order to transfer torque from the motor handpiece to the end effector.

### CROSS-REFERENCE TO RELATED APPLICATIONS

This application is the United States national stage entry of International Application No. PCT/EP2023/071589, filed on Aug. 3, 2023, and claims priority to German Application No. 10 2022 119 979.7, filed on Aug. 9, 2022. The contents of International Application No. PCT/EP2023/071589 and German Application No. 10 2022 119 979.7 are incorporated by reference herein in their entireties.

### FIELD

The disclosure relates to a medical motor handpiece for driving a distal end effector (tool), having a handle portion.

### BACKGROUND

In modern minimally invasive surgery, such tools/instruments and associated instrument handpieces/manual instruments are used, for example, for treating bones, cartilage in arthroscopic interventions, in spinal surgery and similar orthopedic/surgical treatments as well as for processing organic material in neurosurgery. The tools/instruments have a handpiece/handle/handle section and, if applicable, interchangeable effectors, such as milling cutters, rotary knives, a polishing head or similar items. The effector is mounted in a shaft of the tool/instrument at its distal end, possibly rotatably driven. Depending on the intended use and the intended tool speed, a hydraulic, pneumatic or electric motor drive is provided as the tool drive, which is operatively connected to the tool head (effector) via a torque transmission cable within the tool and/or the manual instrument or the handle portion. The drives may be integrated into the tool and/or the manual instrument or may be configured as external drive units that are coupled to the tool or the manual instrument via power supply lines or torque transmission lines.

It is advantageous to angle the distal shaft portion of a shaft of a medical manual instrument in order to be able to perform operations in a small space, for example in operations on the spine. In other words, the installation space of the instruments used and good manageability play a major role in surgical, in particular minimally invasive, interventions. In particular, the instrument shafts in the area of the distal effector should be able to be actively angled (deliberately executed via an actuating mechanism) in a space that is as small as possible.

It is furthermore advantageous if the tools or effectors can be replaced quickly and easily to ensure rapid adaptation to changing operating conditions and/or working environments and to minimize the strain on the patient.

Angleable shafts for medical manual instruments are well known and usually have a proximal and an angleable distal shaft portion. The distal shaft portion and the proximal shaft portion each have an oblique end/an angled front side with respect to the respective shaft portion axis. This means that one end/end portion/front side of the distal and proximal shaft portion is not straight, but oblique/angled. The oblique sections/angled end portions/front sides each have the substantially same work angle. Therefore, the oblique sections fit together in such a way that the proximal and distal shaft portions form a straight shaft/a straight tube in a certain relative rotational position. If the distal shaft portion now rotates about its longitudinal axis relative to the proximal shaft portion and the proximal shaft portion remains stationary, the distal shaft portion is inevitably angled by the angled front sides/end portions.

Furthermore, DE 10 2017 010 033 A1 also discloses a medical device with a guide unit which has a guide tube with a longitudinal axis, a proximal first coupling part firmly connected thereto and distally a cylindrical sleeve-shaped swivel head as well as with an actuating tube which is axially movable in the guide tube and connected to the swivel head and which causes the swivel head to swivel via a proximal operating element. For more precise alignment of a distal guide element for a rotatable surgical tool and thus the angular alignment of the working head of such a tool, the operating element can be swiveled about the longitudinal axis and causes the swivel head to swivel with axial displacement of the actuating tube.

Furthermore, U.S. Pat. No. 7,585,300 B2 or U.S. Pat. No. 10,070,872 B2 disclose examples of surgical instruments with a handpiece and a shaft housed in the handpiece, at the distal end of which a tool head is pivotably articulated. The pivoting of the tool head can be effected by a handwheel rotatably arranged on the handpiece. Similarly, U.S. Pat. No. 8,303,594 B2 discloses a surgical handpiece in which the pivoting of the tool head is effected by a lever arranged on the handpiece.

In addition, U.S. Pat. No. 9,597,093 B2 shows a tool whose proximal end can be coupled to a drive unit in a torque-transmitting manner and which has a tool head at its distal end. The tool head can be pivoted relative to a tool shaft by rotating a sleeve arranged in the area of the tool head.

Further examples of surgical manual instruments with rotating tools that can be angled/swiveled relative to a handpiece are disclosed in U.S. Pat. Nos. 10,178,998 B2, 10,307,180 B2 or U.S. Pat. No. 10,524,820 B2, inter alia.

However, the state of the art always has the disadvantage that the tools with their angleable shafts require complex coupling. Simple and quick decoupling is therefore not easily possible.

### SUMMARY

The objects and objectives of the disclosure are to eliminate or at least reduce the disadvantages of the prior art and in particular to provide an intuitive motor handpiece or manual instrument which allows a simple and quick tool change and at the same time allows a relative movement of the tool during operation.

The objects and objectives are solved with respect to a medical motor handpiece according to the disclosure. The disclosure is thus based on the realization of an integration of two functions in one operating element.

According to the disclosure, the medical motor handpiece is characterized by a preferably sleeve-shaped operating element which is held rotatably about a longitudinal handle axis on a distal end portion of the handle portion and is coupleable to the end effector in such a way as to transform a rotational movement of the operating element in a first direction of rotation into a movement of the end effector and, for a rotational movement of the operating element in a second direction of rotation opposite to the first direction of rotation, to effect a function at the end effector.

In other words, two different functions can be implemented with one rotatable operating element depending on the direction of rotation. Such a 2-in-1 operation of the motor handpiece means that no additional operating element is required, which in turn leads to a more intuitive operation.

### ADVANTAGEOUS EMBODIMENTS ARE EXPLAINED BELOW.

In a preferred variant, the motor handpiece may be coupleable to the end effector via a shaft, and the operating element can cause the end effector to be angled relative to the shaft during a rotational movement of the operating element in the first direction of rotation.

Furthermore, the end effector may preferably have a first coupling device and the shaft may have a second coupling device, which are in operational engagement with each other in a coupling state, wherein a rotational movement of the operating element in the second direction of rotation releases the operational engagement between the first coupling device and the second coupling device.

According to the disclosure, the motor handpiece may be configured according to an advantageous embodiment such that, in a plan view of the end effector, the first direction of rotation is a clockwise direction of rotation and the second direction of rotation is a counterclockwise direction of rotation.

In addition, it may be advantageous if the handle portion and/or the operating element has at least one indicator, preferably in the form of a numerical scale and/or a pictogram, to indicate the movement of the end effector and/or the function.

According to a preferred further development, at least one latching and/or stop unit (locking device) may be provided, in particular in the form of a ball pressure element, which is provided and configured to limit the rotational movement of the operating element at end positions and/or to increase a rotational movement resistance on the operating element in at least one intermediate rotary position.

An advantageous embodiment according to the disclosure may also be characterized by a locking unit, which locks a rotational movement of the operating element in the first direction of rotation and in the second direction of rotation in a locking position and unlocks it in a release position. The locking unit may preferably be configured as a locking slide, which is arranged on the operating element in order to move axially between the locking position and the release position. In addition, it may be particularly advantageous if the locking slide has a spring element that pushes the locking slide into the locking position.

Particularly preferably, the latching and/or stop unit may be configured in the form of at least one ball pressure element and may have a spring-force setting element, preferably in the form of a setscrew, for steplessly variable adjustment of a spring force of the ball pressure element.

In an advantageous embodiment, the motor handpiece may be coupleable to the end effector via a shaft, and the shaft may be coupleable to a rotary transmission sleeve of the motor handpiece in a rotationally fixed manner. It may also be preferable if the operating element transmits its rotational movement to the rotary transmission sleeve via a radial adjusting pin.

According to an advantageous further development, the motor handpiece may have a connection at a proximal end portion for coupling the motor handpiece to a drive unit and a locking unit, which is connected to the operating element in such a way that rotation of the operating element in the second direction of rotation is locked when the motor handpiece is coupled to the drive unit. Preferably, the locking unit may have a locking bolt that is coupled to the operating element and shifts in a proximal direction when the operating element rotates in the second direction of rotation. The drive unit may also form a stop for this purpose, so that a movement of the locking bolt in the coupled state of the motor handpiece and the drive unit is blocked/locked by the drive unit.

Furthermore, the disclosure relates to a medical manual instrument comprising a medical motor handpiece according to the disclosure and an end effector coupled to the motor handpiece via a shaft to transmit torque from the motor handpiece to the end effector.

In other words, the disclosure relates to a (motor) handpiece with a rotary operating unit with which the tool ejection may be initiated and the angulation of the distal tip may be adjusted in several steps. A safety function (ON/OFF) ensures, as with known handpieces, that the tool cannot be accidentally ejected in the ON state.

### BRIEF DESCRIPTION OF THE DRAWINGS

The disclosure is explained in more detail below with reference to preferred configuration examples with the aid of Figures.

FIG. 1 shows a perspective view of a medical manual instrument according to a first embodiment of the present disclosure;

FIG. 2 shows a further perspective view of the medical manual instrument according to the first embodiment of the present disclosure;

FIG. 3 shows a partial longitudinal sectional view of a motor handpiece of the medical manual instrument according to the first embodiment;

FIG. 4 shows a cross-sectional view of the motor handpiece of the medical manual instrument according to the first embodiment;

FIG. 5 shows a partial longitudinal sectional view of a distal end portion of the medical manual instrument according to the first embodiment in a straight shaft shape;

FIG. 6 shows a partial longitudinal sectional view of the distal end portion of the medical manual instrument according to the first embodiment in an angled shaft shape;

FIG. 7 shows a partial longitudinal sectional view of the distal end portion of the medical manual instrument according to the first embodiment in a release state;

FIG. 8 shows an isometric perspective view of the motor handpiece of the medical manual instrument according to the first embodiment;

FIG. 9 shows a partial longitudinal sectional view of the motor handpiece of the medical manual instrument according to the first embodiment;

FIG. 10 shows a perspective longitudinal sectional view of the medical manual instrument according to a modification of the first embodiment;

FIG. 11 shows a detailed view of a sliding motion link of the medical manual instrument according to the modification of the first embodiment;

FIG. 12 shows a perspective view of the medical manual instrument according to the first embodiment in an operating state;

FIG. 13 shows a perspective view of the medical manual instrument according to the first embodiment in an operating state;

FIG. 14 shows an isometric perspective view of a motor handpiece of a medical manual instrument according to a second embodiment;

FIG. 15 shows a longitudinal sectional view of the medical manual instrument according to the second embodiment; and

FIG. 16 shows a cross-sectional view of the medical manual instrument according to the second embodiment.

Figures are schematic in nature and are provided solely for the purpose of understanding the disclosure. Identical elements are provided with the same reference signs. The features of the various configuration examples can be interchanged.

### DETAILED DESCRIPTION

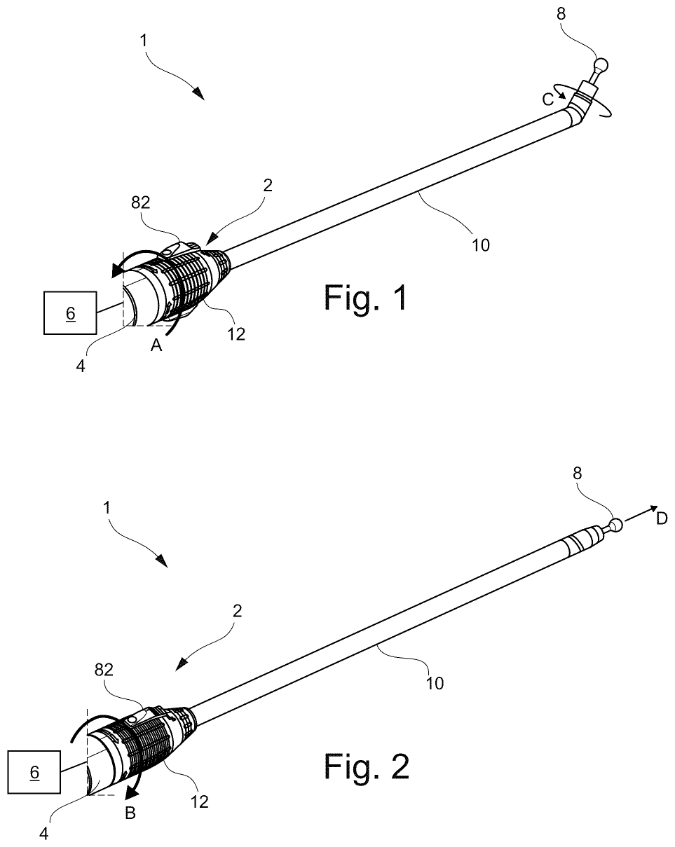

FIG. 1 shows a perspective view of a medical manual instrument 1 according to a first embodiment of the present disclosure. The medical manual instrument 1 has a medical motor handpiece 2, which has a proximal handle portion 4 and which, as shown schematically in FIG. 1, may be coupled to a drive unit 6. Alternatively, the drive unit 6 may also be housed in the motor handpiece 2 and supplied with energy via a power supply connection.

Furthermore, the manual instrument 1 has a distal tool (end effector/effector section) 8, which may be coupled to the motor handpiece 2, in particular to the handle portion 4, via a (tool) shaft 10 or may be decoupled from it. The tool 8 may, for example, be configured as a milling cutter, drill or polishing head.

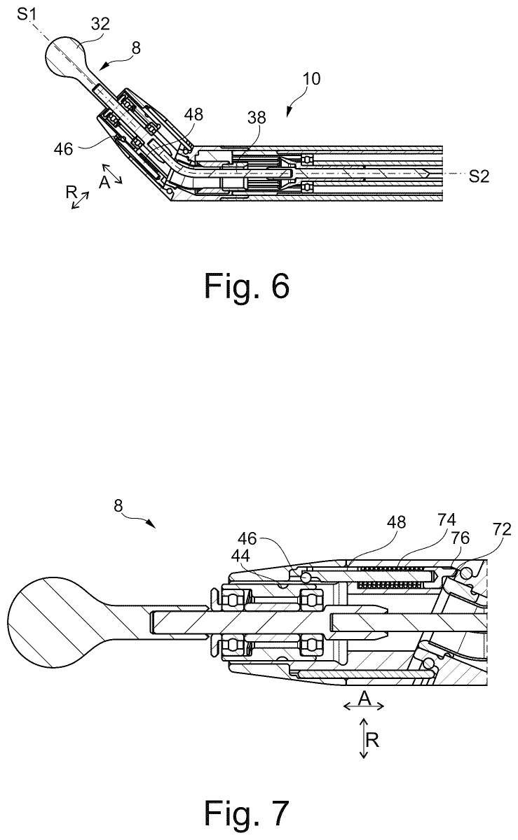

When the shaft 10 is coupled to the handle portion 4, torque is transmitted from the drive unit 6 via a torque transmission cable arranged in the manual instrument 1, in particular the handle portion 4 and the shaft 10, to the tool 8 in order to set it in rotation. When using the manual instrument 1 in the context of a surgical operation or medical treatment, it is known that the tool 8 may be angled relative to the shaft 10 (indicated by arrow C in FIG. 1). In other words, the tool 8 and the shaft 10 may be angled relative to each other, as described in more detail below, in such a way that a longitudinal axis S1 of the tool 8 and a longitudinal shaft axis S2 of the shaft 10 form an angle not equal to 180°, in particular less than 180°(see FIG. 6).

As shown in FIG. 1, a sleeve-shaped operating element 12 is provided for this purpose in the manual instrument 1 according to the disclosure. The operating element 12 is arranged at a distal end portion of the handle portion 4 and is rotatable about a longitudinal handle axis in a first direction of rotation A and in a second direction of rotation B opposite to the first direction of rotation A. In other words, the motor handpiece 2 has the handle portion 4 and the distally arranged operating element 12, which are arranged coaxially to each other, wherein the operating element 12 may be rotated relative to the handle portion 4.

A rotation of the operating element 12 from a neutral zero position, in which the tool 8 is not angled relative to the shaft 10, in the first direction of rotation A causes the tool 8 to be angled relative to the shaft 10, as shown in FIG. 1. In the manual instrument 1 according to the present disclosure, the first direction of rotation A is defined as a rotation of the operating element 12 relative to the handle portion 4 to the left. In other words, the first direction of rotation A corresponds to a clockwise rotation of the operating element 12 in a distal plan view of the tool 8.

As shown in FIG. 2, the operating element 12 in the manual instrument 1 according to the disclosure may also be rotated in the second direction of rotation B, which is opposite to the first direction of rotation A, relative to the handle portion 4. In other words, the second direction of rotation B is defined in the plan view of the tool 8 as a counterclockwise rotation of the operating element 12 or as a rotation to the right. As indicated by the arrow D in FIG. 2, a rotation of the operating element 12 in the second direction of rotation B causes a release of the coupling of the tool 8 with the shaft 10. Thus, as explained in more detail below, a simple and fast tool change can be ensured by a rotation of the operating element 12 in the second direction of rotation B.

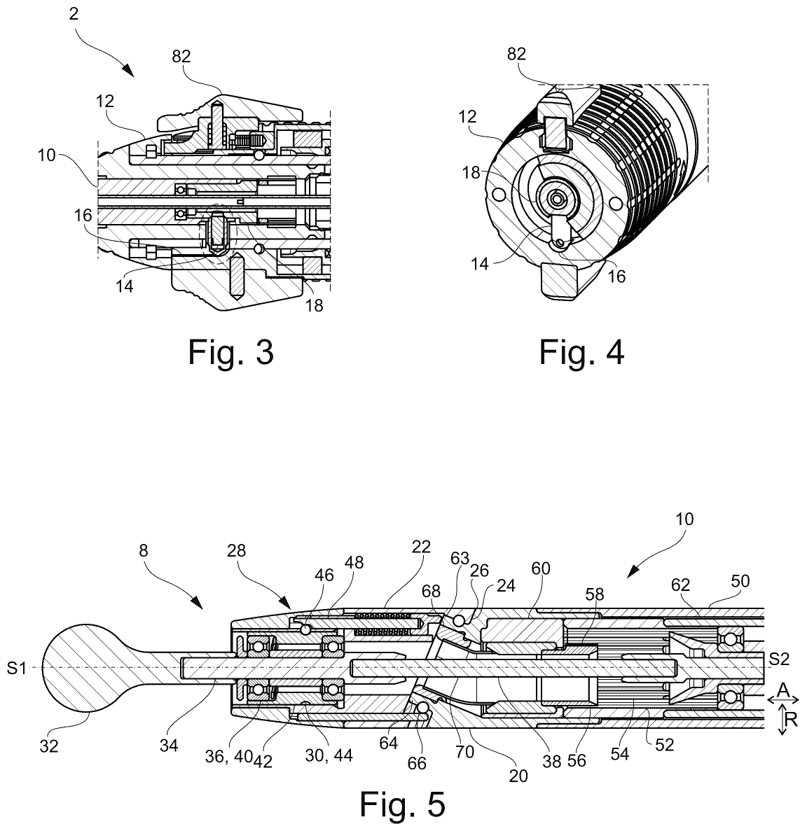

FIGS. 3 and 4 show partial sectional views of the manual instrument 1 according to the first embodiment. In order to transmit the rotational movement of the operating element 12 to the shaft 10, an adjusting pin 14 is arranged in the radial direction of the operating element 12. A radially outer end portion of the adjusting pin 14 is housed in an axial groove 16, which is configured on an inner circumferential surface of the operating element 12. On a radially inner side, the adjusting pin 14 is housed in a rotary transmission sleeve 18. The rotary transmission sleeve 18 is non-rotatably connected to the shaft 10, so that a movement of the adjusting pin 14 in circumferential direction causes the rotary transmission sleeve 18 and the tool 8 to rotate. In other words, when the operating element 12 is rotated in the first direction of rotation A or in the second direction of rotation B, the adjusting pin 14 moves with the operating element 12 in the axial groove 16 and, depending on the direction of rotation, causes the tool 8 to be angled or ejected.

FIG. 5 shows a longitudinal cross-section of a distal end portion of the manual instrument 1 in a straight shaft shape. This means that the operating element 12 is not rotated relative to the handle portion 4 (zero position). As a result, the tool 8 and the shaft 10 are not angled and the longitudinal axis S1 of the tool 8 is collinear to the longitudinal shaft axis S2. As can be seen in FIG. 5, the shaft 10 has a proximal shaft portion 20, which faces the handle portion 4 and may be coupled to it, and a distal shaft portion 22, which may be coupled to the tool 8. When the operating element 12 is not rotated relative to the handle portion 4 and the shaft 10 is in the straight shaft form, the proximal shaft portion 20 and the distal shaft portion 22 are therefore arranged in a line or have an angle of 0°to each other. The proximal shaft portion 20 is substantially tube-shaped and has a front side 24 angled in the longitudinal shaft axis S2 at its distal end portion. The distal shaft portion 22 is also approximately tube-shaped. The tube tapers towards the distal end. The distal shaft portion 22 has a front side 26 angled in the longitudinal shaft axis S2 at its proximal end portion.

The angled front sides 24, 26 each have a work angle of preferably 22.5°to a normal plane to the longitudinal shaft axis S2. When the shaft 10 is in a straight shaft shape or extended, the two angled front sides 24, 26 are offset from each other in such a way that the long ends of the angled front sides 24, 26 are opposite each other in relation to the longitudinal shaft axis S2. The angled front sides 24, 26 lie on top of each other. The angled front sides 24, 26 do not necessarily have to have a work angle of 22.5°. Work angles of, for example, 10°, 18°, 30°, 45°or any other work angle are also conceivable.

As mentioned above, the distal shaft portion 22 is configured to be coupled to the tool 8. That is, the distal shaft portion 22 represents a tool holder, wherein the tool 8 is received in the tool holder and is rotatable relative to the tool holder, i.e. to the distal shaft portion 22.

For this purpose, a first coupling device 28 is arranged in the distal shaft portion 22. A second coupling device 30 is provided on the tool 8, which in a coupling state realizes coupling between the tool 8 and the distal shaft portion 22 by interacting with the first coupling device 28 in order to fix the tool 8 in the distal shaft portion 22 in the axial direction A.

As mentioned above, the longitudinal axis S1 of the tool 8 or of the distal shaft portion 22 and the longitudinal shaft axis S2, i.e. the longitudinal axis of the proximal shaft portion 20 in FIG. 5, form an angle of 0°. In addition, FIG. 5 shows a coupling state between the tool 8 and the distal shaft portion 22. This means that the first coupling device 28, which is mounted in/on the distal shaft portion 22, and the second coupling device 30, which is provided on the tool 8, are in engagement or interact with each other. The tool 8 has a tool head/effector 32, for example a milling head, and a tool shaft 34. The tool head 32 and the tool shaft 34 are non-rotatably connected to each other.

FIG. 5 also shows that the tool shaft 34 is rotatably mounted in the distal shaft portion 22 via a roller bearing unit 36. A drive shaft 38 extends through the proximal shaft portion 20 and is non-rotatably connected to the tool shaft 34 and applies torque from the drive unit 6 to the tool shaft 34 in the form of a torque transmission cable. As a result of this torque application, the tool shaft 34 rotates relative to the distal shaft portion 22. The roller bearing unit 36 has at least one, in this case exactly two, roller bearings 40, more precisely ball bearings, which are spaced apart from each other in the axial direction A, but which may alternatively also be configured as plain bearings. The roller bearings 40 are housed in a bearing housing 42, which is part of the roller bearing unit 36.

The second coupling device 30 is inserted into the bearing housing 42 and is therefore not directly provided on the tool 8. The roller bearing unit 36 and thus also the bearing housing 42 is fixed to the tool shaft 34 in the axial direction A. Alternatively, however, it would also be conceivable that the second coupling device 30 is provided directly on the tool shaft 34. The second coupling device 30 is configured as an axial locking groove 44 that extends continuously in the circumferential direction of the bearing housing 42. The axial locking groove 44 preferably has a semi-circular or circular segment-shaped cross-section.

The first coupling device 28, which is provided in the distal shaft portion 22, has at least one locking ball 46. The diameter of the locking ball 46 is selected such that the locking ball 46 may be housed in the axial locking groove 44. Preferably, the axial locking groove 44 fully surrounds at least one portion of the locking ball 46 contacting the axial locking groove 44. In the coupling state, the locking ball 46 is held in the axial locking groove 44 on its side opposite the axial locking groove 44 by a distal end of a plunger pin/slider 48. The plunger pin 48 is part of the first coupling device 28. The plunger pin 48 is fixed in the radial direction R. The plunger pin 48 is displaceable or movable in the axial direction in the distal shaft portion 22.

In the coupling state shown in FIG. 5, the locking ball 46 is therefore housed in the axial locking groove 44 and is held in the axial locking groove 44 in the radial direction by the plunger pin 48. This position of the plunger pin 48 in the axial direction A is referred to as the coupling position. An edge 63 at the distal end of the proximal shaft portion 20 prevents movement of the plunger pin 48 in the axial direction toward the proximal shaft portion 20.

The proximal shaft portion 20 has a fixed outer pipe 50, a hollow wheel 52 with internal teeth 54, a pinion 56 with outer teeth 58, and an eccentric locking slug 60. The hollow wheel 52 is positioned inside the outer pipe 50 and the longitudinal axis of the outer pipe 50 corresponds to the longitudinal axis of the hollow wheel 52. The outer pipe 50 and the hollow wheel 52 are thus arranged concentrically. The hollow wheel 52 is connected to the rotary transmission sleeve 18, i.e. to the operating element 12, via a hollow shaft 62 received in the proximal shaft portion 20.

As mentioned above, the outer pipe 50 is designed as a fixed pipe and therefore does not move. The distal end of the outer pipe 50 has the angled front side 24. The distal end of the outer pipe 50 also has a receiving bore 64 and a receiving pin for a roller bearing 66. The outer pipe 50 has a notch/groove for the ball of the roller bearing 66. The distal shaft portion 22 is mounted on the roller bearing 66.

The internal teeth 54 mesh with the outer teeth 58 of the pinion 56. As a result, the rotation of the hollow wheel 52, which is controlled by the operating element 12, is transmitted to the pinion 56. The direction of rotation of the pinion 56 is the same as the direction of rotation of the hollow wheel 52. The pinion 56 is driven by the hollow wheel 52, but the pinion 56 rotates in the eccentric locking slug 60. The locking slug 60 is arranged eccentrically to the hollow wheel 52. That is, although the longitudinal axis of the eccentric locking slug 60 is parallel to the longitudinal axis of the hollow wheel 52, the longitudinal axes are not superimposed or offset relative to each other. The distal shaft portion 22 has an adjustment bushing 68. The adjustment bushing 68 is mounted in the receiving bore 64 of the proximal shaft portion 20. The adjustment bushing 68 is connected to the pinion 56 via a flexible silicone hose 70 in such a way that rotation of the pinion 56 is transmitted to the adjustment bushing 68. For this purpose, the flexible silicone hose 70 is attached to the adjustment bushing 68 and the pinion 56, for example by welding or gluing. The adjustment bushing 68 is also connected to the distal shaft portion 22 in a form-fitting manner via a follower pivot (not shown), so that rotation of the adjustment bushing 68 is transmitted to the distal shaft portion 22.

FIG. 6 shows a longitudinal cross-section through the distal end portion of the shaft 10, wherein the distal shaft portion 22 is angled by 22.5°to the proximal shaft portion 20 at a respective work angle of the two front sides 24, 26, i.e. by 45°. Compared to the position in FIG. 5, the distal shaft portion 22 is rotated by 180°around its own longitudinal axis S1. In this position, the angled front sides 24, 26 lie completely/flat on top of each other again. Due to the rotation of the distal shaft portion 24, however, the long ends of the angled front sides 24, 26 are positioned next to each other. The work angles of the angled front sides 24, 26 thus add up. As a result, the distal shaft portion 22 is angled by twice the working angle of the angled front sides 24, 26 compared to the proximal shaft portion 20.

In the position shown in FIG. 6, the follower pivot is arranged opposite the locking slug 60. This means that the adjustment bushing 68 has rotated 180°from the extended position to the maximum angled position. The 45°position represents the reversal point for this design. The adjustment bushing 68 has rotated by 180°in this position. With further rotation of the hollow wheel 52, i.e. the operating element 12, the distal shaft portion 22 would rotate back to the initial position (zero position).

Furthermore, it can be seen in FIG. 6 that the locking ball 46 is held in the axial locking groove 44 by the plunger pin 48 in the radial direction R even when the distal shaft portion 22 is angled in the maximum adjustable angular position or maximum angular deflection with respect to the proximal shaft portion 20. In this way, the tool 8 is secured in the distal shaft portion by the engagement between the first coupling device 28 and the second coupling device 30, even in this angular position in the axial direction A.

The torque string, in particular the driveshaft 38, toward the tool shaft 34, which is arranged in the transition region between the distal shaft portion 22 and the proximal shaft portion 20, is flexible. This flexible portion of the torque string allows the distal portion of the tool shaft 34 with tool head 32 to be angled together with the distal shaft portion 22 relative to the torque string in the proximal shaft portion 20. At the same time, the flexible portion of the torque string is designed such that it can continue to transmit a torque exerted on a proximal portion of the tool shaft 34 to the tool head 32.

FIG. 7 is a longitudinal sectional view of the distal end portion of the shaft 10 in a release state between the tool 8 and the distal shaft portion 22. I.e. in the position shown in FIG. 7, the first coupling device 28 and of the second coupling device 30 are not in operational engagement with each other, so that the tool 8 may be removed or changed. In order to release the operational engagement of the first coupling device 28 and the second coupling device 30, the operating element 12 is rotated in the second direction of rotation B (see FIG. 2). As explained in more detail below, the locking ball 46 is no longer housed in the axial locking groove 44. Instead, it is held by the plunger pin 48 in the radial direction R against an outer circumferential surface of the bearing housing 42.

In order for the locking ball 46 to move from the coupling state shown in FIG. 5 out of the axial locking groove 44 into the release state, the plunger pin 48 has to move in the axial direction A toward the proximal shaft portion 20. In the coupling state, this is prevented by the circumferential edge 63 of the proximal shaft portion 20. However, the edge 63 is interrupted at one point of a plunger-pin receiving recess 72. When the operating element 12 is rotated in the second direction of rotation B, the distal shaft portion 22 rotates relative to the proximal shaft portion 20 in a direction opposite to the angling, for example by (−)18°, until the plunger pin 48 is positioned relative to the proximal shaft portion 20 such that it is level with the plunger-pin receiving recess 72 in the circumferential direction. A pretensioning element 74, which is part of the first coupling device 28, pushes the plunger pin 48 in the axial direction A toward the proximal shaft portion 20. The pretensioning element 74 thus pushes the plunger pin 48, or more precisely its proximal end, which is configured as a latching projection 76, into the plunger-pin receiving recess 72. The position in which the plunger pin 48 is when its latching projection 76 engages in the plunger-pin receiving recess 72 is referred to as the release state or release position.

In the release position, the distal end of the plunger pin 48 is no longer opposite the axial locking groove 44. This means that the locking ball 46 is not held in the axial locking groove 44 in the radial direction R. The tool 8 is therefore no longer fixed relative to the distal shaft portion 22 in the axial direction A. If, starting from the coupling state, a tensile force (in the axial direction A) is now applied to the distal end of the tool 8, the locking ball 46 is released from the axial locking groove 44. In this way, the tool 8 can be uncoupled from the distal shaft portion 22.

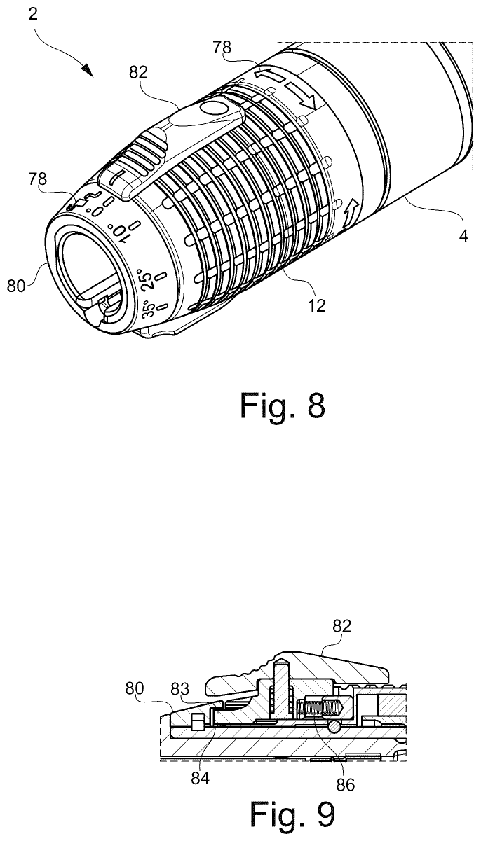

FIG. 8 shows a perspective view of the distal end portion of the motor handpiece 2 according to the first embodiment, without the shaft 10 being coupled to the handle portion 4. As described above, a rotation of the operating element 12 in the first direction of rotation A causes the tool 8 to be angled and a rotation of the operating element 12 in the second direction of rotation B releases the coupling between the tool 8 and the shaft 10, or the distal shaft portion 22, by releasing the operational engagement between the first coupling device 28 and the second coupling device 30. In order to facilitate operation, indicators 78 in the form of arrows indicating the directions of rotation are provided on the operating element 12. In combination with indicators 78 on an indicator sleeve 80, which is rotationally fixed to the handle portion 4 distal to the operating element 12, the user can quickly recognize which direction of rotation corresponds to which function during operation of the manual instrument 1. For this purpose, as shown in FIG. 8, various angular positions are applied to an outer circumferential surface of the indicator sleeve 80, each of which corresponds to a defined rotation of the operating element 12 in the first direction of rotation A and thus a defined angling of the tool 8 relative to the shaft 10, as well as a lock symbol or a symbol of an open lock. The user can thus assign the respective function, i.e. angling or uncoupling, to the individual directions of rotation A, B. The motor handpiece 2 together with the operating element 12 thus enables intuitive operation and integration of the two functions.

As shown in FIG. 8, a locking slide 82 is arranged on the motor handpiece 2 according to the first embodiment. The locking slide 82 is fixedly received in the operating element 12 in the radial and circumferential directions in such a way that the locking slide 82 can only move in the axial direction relative to the operating element 12 between a (distal) locking position and a (proximal) release position. In the locking position, the operating element 12 cannot be rotated relative to the handle portion 4, as explained in more detail below.

For this purpose, in the locking position, a distal tappet portion 83 of the locking slide 82, as shown in FIG. 9, engages with a locking ring 84 received in the indicator sleeve 80. The locking ring 84 has a plurality of recesses distributed over the circumference, into which the tappet portion 83 projects in the locking position, in order to fix or hold the operating element 12 in the circumferential direction relative to the handle portion 4. The recesses of the locking ring 84 preferably correspond to the indicators 78 attached to the outer circumferential surface of the indicator sleeve 80. Thus, the user can easily set the tool 8 to be angled at a defined angle and fix the angled tool 8 at this angle.

In order to be able to rotate the operating element 12 relative to the handle portion 4 to adjust the angle or to release the clutch, the locking slide 82 has therefore to be moved from the locking position to the release position, i.e. in the proximal direction.

In the motor handpiece 2 according to the first embodiment, the locking slide 82 is pretensioned in the axial direction against the operating element 12 via a spring element 86. The spring element 86 is arranged between the locking slide 82 and a stop surface of the operating element 12 in such a way that it presses the locking slide 82 into the locking position, i.e. in the distal direction. The spring element 86 thus exerts an automatic restoring force on the locking slide 82 in order to hold it in the locking position.

Alternatively, it is of course also conceivable that the motor handpiece 2 does not have a spring element 86. In such a motor handpiece according to a modification of the first embodiment with manual reset, the user has to pull the locking slide 82 in the proximal direction for unlocking and has to actively push it in the distal direction for locking.

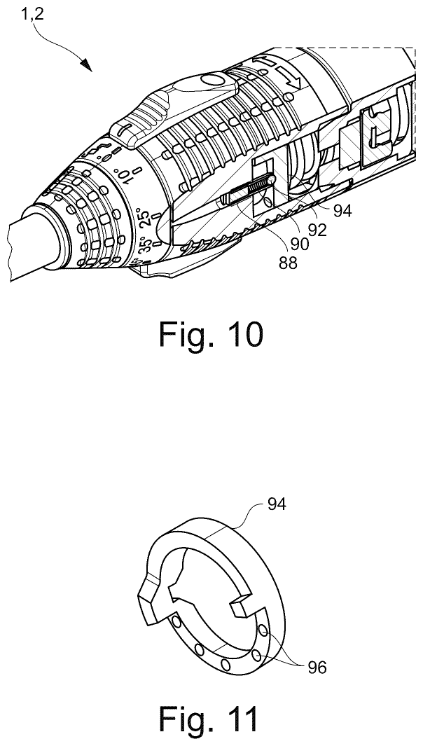

As shown in FIGS. 10 and 11, the motor handpiece 2 according to the modification of the first embodiment has an additional ball pressure element 88 provided. This has a spring element 90, which presses a ball 92 in the proximal direction against a sliding motion link (calotte latching ring) 94 (see FIG. 11), which is firmly held in the handle portion 4. Calotte-shaped latching recesses 96 are configured on the sliding motion link 94, which may at least partially accommodate the ball 92.

Similar to the recesses of the locking ring 84, the latching recesses 96 correspond to defined angles by which the tool 8 may be angled relative to the shaft 10 when the operating element 12 is rotated by a certain angle. I.e., when the operating element 12 is rotated by the defined angle relative to the handle portion 4, the ball pressure element 88 rotates with the operating element 12 until the defined angle is reached at which the ball 92 engages in the corresponding latching recess 96 of the sliding motion link 94 due to the preload force of the spring element 90. The additional ball pressure element 88 therefore provides haptic feedback for the user when adjusting the angle.

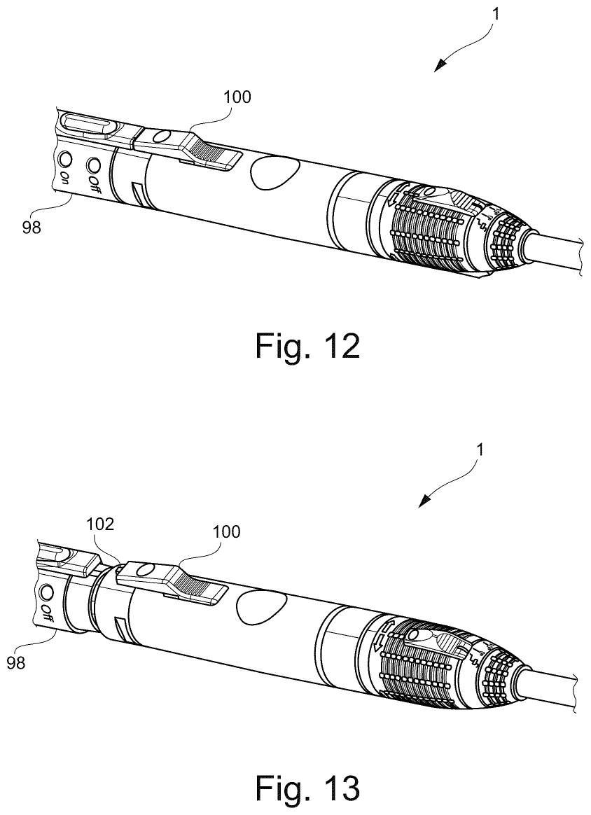

FIG. 12 shows the medical handpiece 1 according to the first embodiment in an operating state in which the motor handpiece 2 is connected to the drive unit 6. For this purpose, as mentioned above, the motor handpiece 2 has, at its proximal end portion, the connection which, as shown in FIG. 12, may accommodate a motor cable 98. The motor handpiece 2 also has a stop slider 100, which may be moved proximally in the OFF position (see FIG. 13) in order to release the connection between the shaft 10 and the motor handpiece 2.

In order to secure the release position of the tool 8, the stop slider 100 has a locking bolt 102, as can be seen in FIG. 13. The locking bolt 102 is connected to the operating element 12 and is extended in the proximal direction when the tool 8 is unlocked by turning the operating element 12 in the second direction of rotation B, i.e. when the operational engagement between the first coupling device 28 and the second coupling device 30 is released.

When the medical handpiece 1 is in the operating state, this proximal extension of the locking bolt 102 is not possible, since the motor cable 98, in particular a lug of the motor cable 98, blocks an extension. This prevents the tool 8 from being ejected during operation if the operating element 12 is inadvertently turned in the second direction of rotation B. However, it is still possible to rotate the operating element 12 in the first direction of rotation A, so that the tool 8 can also be angled relative to the shaft 10 during operation.

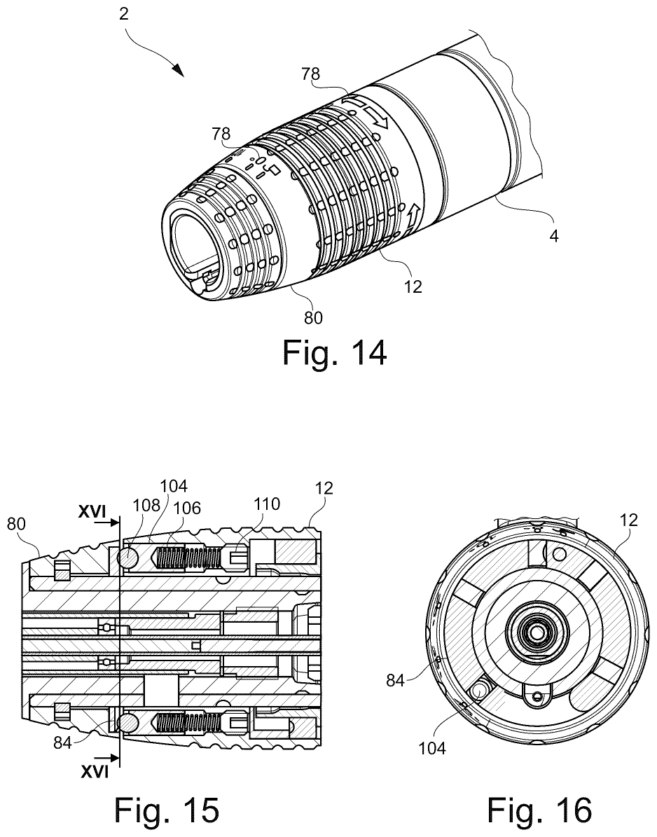

FIG. 14 shows a motor handpiece 2 for a medical manual instrument 1 according to a second embodiment. It can be seen that the motor handpiece 2 according to the second embodiment does not have a locking slide 82. The omission of the locking slide 82 enables an improved view of the operating field, more intuitive operation and reduced cleaning effort.

In order to nevertheless ensure a certain degree of protection of the operating element 12 against unintentional rotation, even without locking slide 82, and to enable the operating element 12 to be locked at defined angles, two ball pressure elements 104 are arranged in the operating element 12, as shown in FIG. 15. These each have a spring element 106, which presses a ball 108 as a latching body against the locking ring (latching plate) 84. I.e., the spring element 106 presses the ball 108 in the distal direction against the locking ring 84, which, as can be seen in FIG. 16, has recesses with which the balls 108 latch.

As mentioned above, the ball pressure elements 104 press with their distal end portions, i.e. the balls 108, against the locking ring 84. At their proximal end portion, the ball pressure elements 104 each have a spring force adjustment means in the form of a setscrew 110. By screwing in the setscrew 110, the pre-stressing of the spring element 106 and thus the spring force acting on the locking ring 84 may be adjusted. A higher spring force makes it more difficult to accidentally twist the operating element 12, whereas a lower spring force makes it easier to adjust the angle and thus enables one-handed operation.

### LIST OF REFERENCE SIGNS

1 manual instrument
2 motor handpiece
4 handle portion
6 drive unit
8 tool
10 shaft
12 operating element
14 adjusting pin
16 axial groove
18 rotary transmission sleeve
20 proximal shaft portion
22 distal shaft portion
24, 26 angled front side
28 first coupling device
30 second coupling device
32 tool head/effector
34 tool shaft
36 roller bearing unit
38 driveshaft
40 roller bearing
42 bearing housing
44 axial locking groove
46 locking ball
48 plunger pin
50 outer pipe
52 hollow wheel
54 internal teeth
56 pinion
58 outer teeth
60 locking slug
62 hollow shaft
63 edge of the proximal shaft portion
64 receiving bore
66 roller bearing
68 adjustment bushing
70 silicone hose
72 plunger-pin receiving recess
74 pretensioning element
76 latching projection
78 indicator
80 indicator sleeve
82 locking slide
83 tappet portion
84 locking ring
86 spring element
88 ball pressure element
90 spring element
92 ball
94 sliding motion link (calotte latching ring)
96 latching recess
98 motor cable
100 stop valve
102 locking bolt
104 ball pressure element
106 spring element
108 ball
110 setscrew

1.-10. (canceled)

11. A medical motor handpiece for driving an end effector, the medical motor handpiece comprising:
a handle portion having a distal end portion with a longitudinal handle axis; and
an operating element held rotatably about the longitudinal handle axis on the distal end portion of the handle portion,
the operating element being coupleable to the end effector in such a way as to:
transform rotational movement of the operating element in a first direction of rotation into movement of the end effector and,
effect a function at the end effector in response to rotational movement of the operating element in a second direction of rotation opposite to the first direction of rotation.

12. The medical motor handpiece according to claim 11, wherein:
the medical motor handpiece is coupleable to the end effector via a shaft, and
the operating element causes the end effector to be angled relative to the shaft during rotational movement of the operating element in the first direction of rotation.

13. The medical motor handpiece according to claim 12, wherein:
the end effector comprises a first coupling device and the shaft comprises a second coupling device in an operational engagement with the first coupling device in a coupling state, and
rotational movement of the operating element in the second direction of rotation releases the operational engagement between the first coupling device and the second coupling device.

14. The medical motor handpiece according to claim 11, further comprising at least one latching and/or stop unit configured to limit rotational movement of the operating element at end positions and/or to increase resistance to rotational movement of the operating element in at least one intermediate rotary position.

15. The medical motor handpiece according to claim 14, wherein the at least one latching and/or stop unit comprises at least one ball pressure element and a spring-force setting element for stepless variable-adjustment of a spring force of the at least one ball pressure element.

16. The medical motor handpiece according to claim 11, further comprising a locking unit, wherein:
the locking unit is operable in a locking position to lock the operating element against rotational movement in the first direction of rotation and in the second direction of rotation, and
the locking unit is operable in a release position to unlock the operating element from the locking position and allow rotational movement of the operating element in the first direction of rotation and in the second direction of rotation.

17. The medical motor handpiece according to claim 11, wherein:
the medical motor handpiece is coupleable to the end effector via a shaft, and
the shaft is coupleable to a rotary transmission sleeve of the medical motor handpiece in a rotationally fixed manner.

18. The medical motor handpiece according to claim 17, further comprising a radial adjusting pin, wherein rotational movement of the operating element is transmitted to the rotary transmission sleeve via the radial adjusting pin.

19. The medical motor handpiece according to claim 11, further comprising a proximal end portion and a connection at the proximal end portion, the connection configured to couple the medical motor handpiece to a drive unit and a locking unit, the locking unit being connected to the operating element in such a way that rotation of the operating element in the second direction of rotation is locked when the medical motor handpiece is coupled to the drive unit.

20. A medical manual instrument comprising:
the medical motor handpiece according to claim 11; and
an end effector coupled to the medical motor handpiece via a shaft to transmit torque from the medical motor handpiece to the end effector.
